(19) United States
(12) Patent Application Publication
(10) Pub. No.: US 2015/0094787 A1
(43) Pub. Date: Apr. 2, 2015

(54) METHODS AND MATERIALS FOR TREATING SYNCOPE

(71) Applicant: Mayo foundation for Medical Education and Research, Rochester, MN (US)

(72) Inventors: Malini Madhavan, Rochester, MN (US); Samuel J. Asirvatham, Rochester, MN (US)

(73) Assignee: Mayo Foundation for Medical Education and Research, Rochester, NY (US)

(21) Appl. No.: 14/399,016

(22) PCT Filed: May 8, 2013
(86) PCT No.: PCT/US2013/040180

§ 371 Date: Nov. 5, 2014

Related U.S. Application Data
(60) Provisional application No. 61/644,393, filed on May 8, 2012.

(51) Int. Cl. A61N 1/36

(52) U.S. Cl. CPC A61N 1/3606; A61N 1/36139; A61N 1/0551
USPC 607/62; 607/2

(57) ABSTRACT

This document provides methods and materials for treating syncope (e.g., neurocardiogenic syncope). For example, methods and materials involved in using electrical techniques to stimulate nerves (e.g., renal efferent and/or afferent nerves) in a manner that results in systemic blood vessel constriction and/or increased blood pressure are provided.

### CROSS-REFERENCE TO RELATED APPLICATIONS

This application claims the benefit of U.S. Provisional Application Ser. No. 61/644,393, filed May 8, 2012. The disclosure of the prior application is considered part of (and is incorporated by reference in) the disclosure of this application.

### BACKGROUND

1. Technical Field

This document relates to methods and materials involved in treating syncope (e.g., neurocardiogenic syncope). For example, this document relates to methods and materials involved in using electrical techniques to stimulate nerves (e.g., renal efferent and/or afferent nerves) in a manner that results in systemic blood vessel constriction and/or increased blood pressure.

2. Background Information

Neurocardiogenic syncope is a common condition with a prevalence of about 22% in the U.S. population and accounts for about 20% of new cases of syncope. Recurrence of neurocardiogenic syncope is common (up to 30%) despite current therapy and can result in physical and psychological morbidity in some patients.

### SUMMARY

This document provides methods and materials for treating syncope (e.g., neurocardiogenic syncope). For example, this document provides methods and materials involved in using electrical techniques to stimulate nerves (e.g., renal efferent and/or afferent nerves) in a manner that results in systemic blood vessel constriction and/or increased blood pressure.

As described herein, stimulating renal nerves (e.g., renal efferent or afferent nerves), as opposed to blocking renal nerve signals, can cause systemic blood vessel constriction and/or an increase in blood pressure. Any appropriate electrical technique can be used to stimulate renal nerves in a manner that results in a clinical improvement for a patient suffering from syncope. For example, an implantable electrode device designed to deliver electrical pulses capable of stimulating renal nerves can be positioned within a mammal (e.g., a human) suffering from or prone to suffer from syncope such that the electrode device can stimulate one or more renal nerves. In some cases, such an implantable electrode device can include one or more sensors configured to monitor the patient's blood pressure and/or heart rate. Once a decrease in blood pressure and/or heart rate is detected, the implantable electrode device can stimulate one or more renal nerves in a manner that results in systemic blood vessel constriction and/or increased blood pressure within the patient. In some cases, an implantable electrode device provided herein can include a pacemaker lead positioned in the patient's heart.

In general, one aspect of this document features a method for treating a mammal undergoing syncope. The method comprises, or consists essentially of, stimulating nerves of the mammal under conditions wherein the blood pressure of the mammal increases. The mammal can be a human. The nerves can be autonomic nerves in or around a renal vessel. The nerves can be spinal nerves. The method can comprise stimulating autonomic ganglia.

In another aspect, this document features a method for treating syncope. The method comprises, or consists essentially of, (a) detecting a decrease in blood pressure within a mammal suffering from syncope, and (b) stimulating nerves of the mammal under conditions wherein the blood pressure of the mammal increases. The mammal can be a human. The nerves can be autonomic nerves in or around a renal vessel. The nerves can be spinal nerves. The method can comprise stimulating autonomic ganglia.

In another aspect, this document features an implantable device for treating syncope of a mammal The device comprises, or consists essentially of, a control unit, a sensing lead for detecting the blood pressure or heart rate of the mammal, and a stimulation lead for stimulating nerves of the mammal, wherein the sensing lead and the stimulation lead are connected to the control unit, and wherein the control unit is configured to activate the stimulation lead to stimulate the nerves when the sensing lead detects a decrease in the blood pressure or heart rate of the mammal The mammal can be a human. The nerves can be autonomic nerves in or around a renal vessel. The nerves can be spinal nerves.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a diagram of one example of an implantable electrode device that can be used to treat syncope.

FIG. 2 is a diagram of a mammal's vascular anatomy and an exemplary location of an electrical lead for stimulating renal efferent or afferent nerves.

FIG. 3 is a flow diagram of an exemplary method for monitoring and treating a patient who suffers from syncope.

FIG. 4 is a flow diagram of an exemplary method for monitoring and treating a patient who suffers from syncope.

FIG. 5A is a photograph showing the placement of a pacing catheter in the right renal vein. FIG. 5B is a graph plotting a recording of arterial tracing during stimulation. The blood pressure increased from a baseline of 139/80 to 163/104 following 30 seconds of stimulation.

### DETAILED DESCRIPTION

This document provides methods and materials for stimulating renal nerves to treat syncope. For example, this document provides methods and materials for using electrical techniques to stimulate renal nerves. As described herein, an electrode lead of an electrode device can be used to stimulate renal nerves in a manner that causes an acute increase in blood pressure. For example, electrical techniques can be used to stimulate a renal nerve via an electrode lead located within a vessel of a kidney (e.g., a renal artery or renal vein). In some cases, an electrode lead of an electrode device can be positioned within a renal artery or vein that is richly innervated by post-ganglionic sympathetic fibers such that a stimulation signal can be delivered to the renal nerves in a manner that causes an acute increase in blood pressure.

In some cases, an electrode lead for stimulation can be placed near a spinal nerve that receives signals from the heart or kidneys (e.g., within the T1 to L5 spinal region), can be placed near an autonomic ganglion (e.g., the celiac plexus, dorsal roots, etc.), or can be placed directly on or around the nerves of a renal vessel (e.g., a renal artery or vein). Any appropriate method can be used to position an electrode lead in such a location (e.g., near a spinal nerve). For example, laparoscopic or direct surgical implantation techniques can be used to position an electrode lead directly on or around the nerves of a renal vessel.

Any appropriate electrical technique can be used to stimulate renal nerves. For example, one or more electrode devices can be used to stimulate renal nerves. In some cases, an implantable electrode device can be used to deliver electrical stimulation therapy signals to renal nerves in a manner that stimulates those nerves and results in increased blood pressure. Examples of electrode devices that can be used to deliver electrical stimulation therapy signals to renal nerves include, without limitation, those devices configured to include one or more electrodes as described elsewhere (see, e.g., U.S. Pat. No. 8,010,204, U.S. Pat. No. 7,783,362, U.S. Pat. No. 6,928,320, PCT Publication No. WO/2011/156439, U.S. Patent Application Publication No. 20040172085, U.S. Patent Application Publication No. 20050131485, U.S. Patent Application Publication No. 20070073354, U.S. Patent Application Publication No. 20100191311, U.S. Patent Application Publication No. 20120016448, U.S. Patent Application Publication No. 20100114244, or EP1904160). In some cases, neurostimulator devices such as a Medtronic™ Soletra or Kinetra can be implanted into a mammal and configured to deliver electrical stimulation therapy signals to renal nerves in a manner that stimulates renal nerves and results in increased blood pressure. For example, a lead (or electrode) of a neurostimulator device can be positioned directly within a blood vessel of a kidney (e.g., a renal artery or renal vein) or within about 7.5 mm (e.g., within 7, 6, 5, 4, 3, 2, or 1 mm) of a renal nerve or renal ganglion of interest to provide electrical stimulation therapy signals that result in an increase in blood pressure. In some cases, a battery powered neurostimulator control unit of a neurostimulator device can be implanted (e.g., in the patient's chest) and can have one or more extensions connecting the neurostimulator control unit to one or more electrodes positioned at a targeted renal nerve location (e.g., within a renal vessel or near an autonomic ganglion such as the celiac plexus or dorsal roots). In some cases, one or more electrodes can be positioned unilaterally or bilaterally within a mammal to be treated (e.g., human to be treated). For example, electrodes can be located within the renal veins of each kidney.

In some cases, more than one location within a mammal's body can be targeted to stimulate renal nerves. For example, one or more electrode devices can be implanted into a mammal such that renal nerves are stimulated via an electrode lead located within a vessel of a kidney and via an electrode lead located near spinal nerves that receive signals from the kidneys.

Any appropriate electrical therapy signals can be used provided that they stimulate a renal nerve in a manner that increases blood pressure. For example, electrical stimulation therapy signals can be designed to deliver a stimulatory signal as described in Example 1.

In some cases, an electrode device provided herein (e.g., a percutaneous electrode device) can be configured to stimulate renal nerves, to monitor blood pressure, to monitor heart rate, to perform cardiac pacing, or a combination thereof. For example, electrode device provided herein can be configured to stimulate renal nerves and to monitor a patient's blood pressure and heart rate. In such cases, a sensing lead or pacemaking lead can be positioned within the patient's heart (e.g., within the right vertical) or positioned in another location (e.g., a carotid artery, renal artery, or aorta).

In some cases, an electrode device provided herein (e.g., a percutaneous electrode device) can be configured to perform cardiac pacing and renal nerve stimulation. In such cases, a transvenous renal vein electrode can include multiple electrodes mounted on a nitinol or mesh scaffold that opposes the electrodes to the wall of the vein. The device can include a transvenous right ventricular pacemaker lead with a pacing electrode in the renal vein implanted percutaneously and connected to a device generator. Impending neurocardiogenic syncope can be identified by the device algorithm as a sudden drop in heart rate or blood pressure, which can trigger rapid cardiac pacing and renal nerve stimulation to simultaneously increase the heart rate and blood pressure, thus preventing syncope.

In some cases, an electrode device provided herein can include an expandable multi-electrode basket catheter having multiple electrode pairs mounted (e.g., in parallel) on an expandable nitinol scaffold and a pacing catheter. The multi-electrode basket catheter can be deployed in one or both renal veins through a percutaneous trans-iliac venous approach. In some cases, a multi-electrode basket catheter can be positioned within the renal pelvis region through a trans-ureteral approach. The renal sympathetic nerves around the renal vein can be stimulated by rapid pacing via the multi-electrode basket catheter using a Grass stimulator (e.g., Grass technology). The pacing catheter can be inserted into the right ventricle.

In some cases, an electrode device provided herein (e.g., a percutaneous abdominal electrode device) can be configured to include a transvenously implantable right ventricular lead and renal vein lead implantable though an iliac vein approach. The right ventricular lead can be configured to sense sudden onset bradycardia, and the renal vein catheter, when positioned via the inferior vena cava, can be configured to include a piezoelectric crystal capable of using Doppler technology to detect a fall in blood pressure in the adjacent abdominal aorta. A pre-determined degree of bradycardia and/or hypotension can trigger an event such that the device delivers rapid cardiac pacing and renal nerve stimulation.

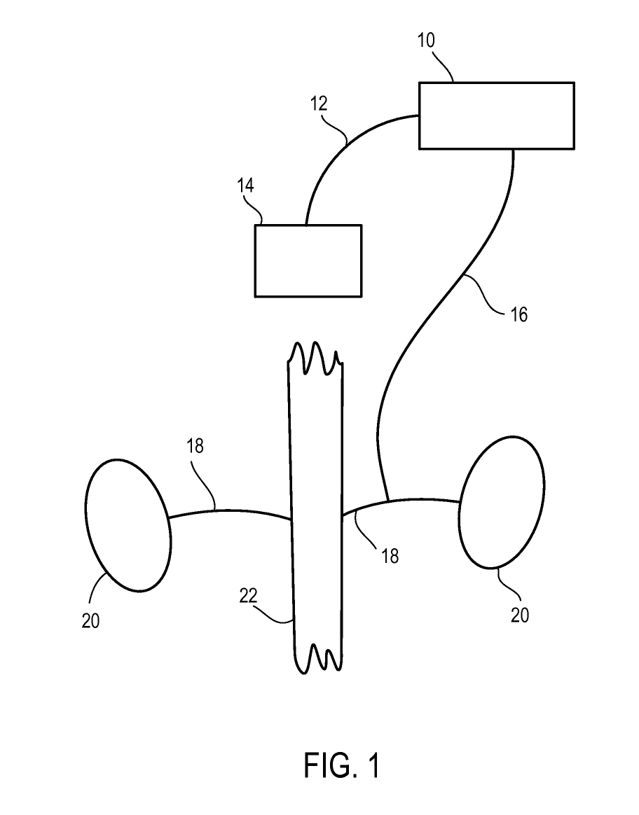

With reference to FIG. 1, an electrode device 10 can be implanted within a mammal (e.g., a human suffering from syncope) with an electrode lead 16 extending to a renal nerve 18. Renal nerve 18 can be a nerve that extends between a kidney 20 to a spinal cord 22. Electrode device 10 can be configured to deliver electrical signals to renal nerve 18 via electrode lead 16 in a manner that results in increased blood pressure. In some cases, electrode device 10 can include a sensing lead 12 configured to monitor blood pressure and/or heart rate. Sensing lead 12 can be positioned within heart 14. For example, sensing lead 12 can be positioned within the right ventricle of heart 14. In some cases, sensing lead 12 can include the ability to function as a pacemaker. For example, sensing lead 12 can be a pacemaker lead configured to monitor blood pressure and heart rate.

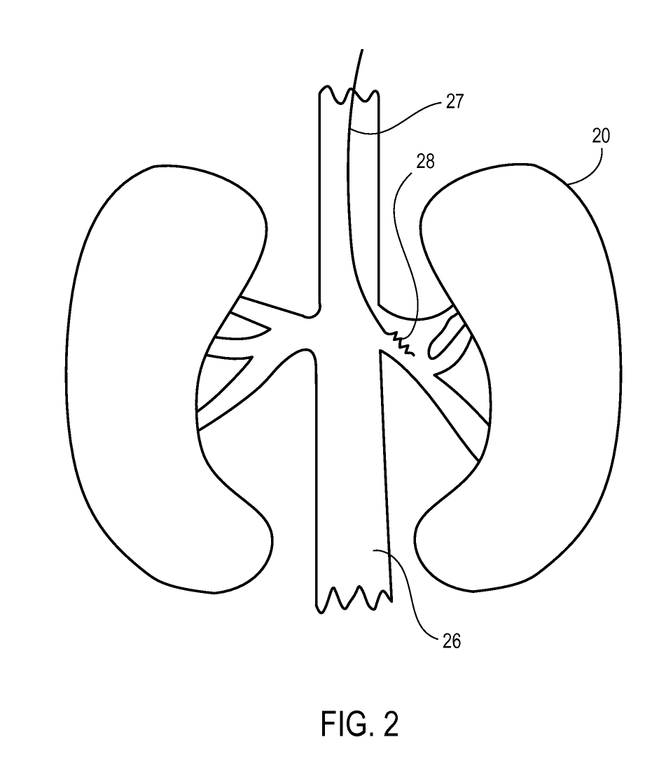

With reference to FIG. 2, an electrode lead 27 of an electrode device (not shown) can be positioned to extend within at least a portion of the aorta or inferior vena cava (either of which is represented using reference number 26) so that an electrode portion 28 of electrode lead 27 is located within a renal blood vessel of kidney 20.

During use, sensing lead 12 of electrode device 10 can be used to detect a reduction in blood pressure. Once this blood pressure reduction is detected, electrode device 10 can send one or more stimulation signals to electrode lead 16 (or electrode lead 27 of FIG. 2) to stimulate renal nerve 18 in a manner the results in an increase in blood pressure. Once sensing lead 12 of electrode device 10 detects a normal blood pressure level, electrode device 10 can stop sending the stimulatory signals to electrode lead 16. In some cases, electrode device 10 can be configured to send one or more pacemaker signals to sensing lead 12 to increase the patient's heart rate. For example, electrode device 10 can be configured to send one or more pacemaker signals to sensing lead 12 to increase the patient's heart rate during the period of time that sensing lead 12 detects a reduced blood pressure level.

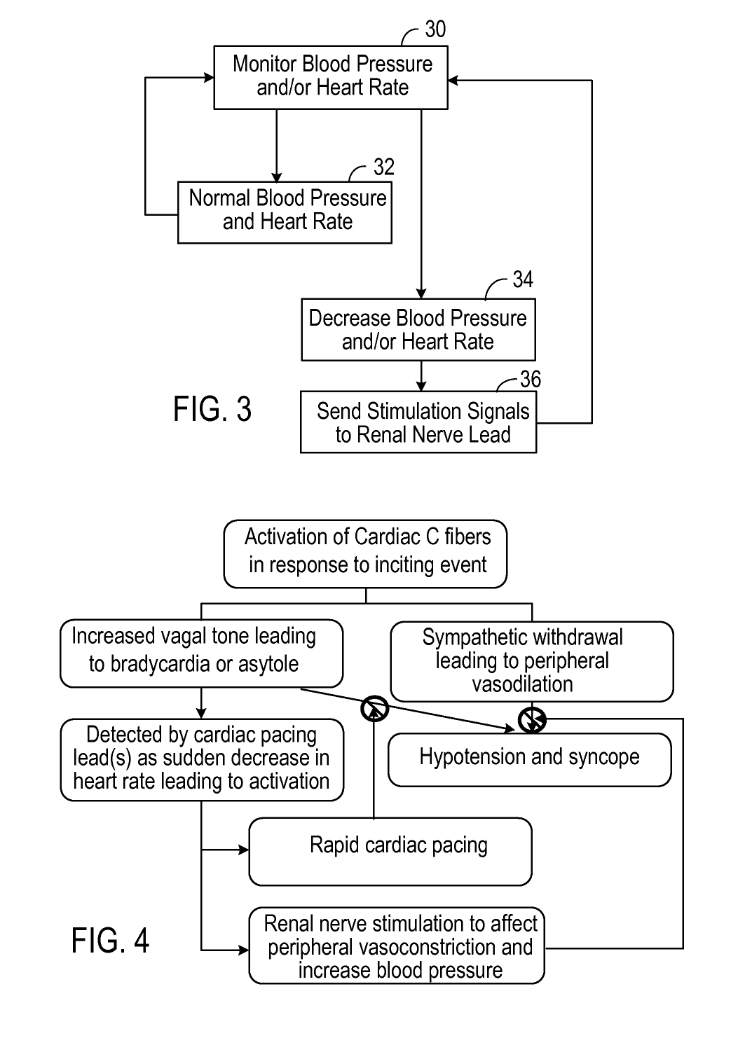

In some cases, a mammal suffering from syncope can be treated using an electrode device provided herein as set forth in FIG. 3. For example, an electrode device provided herein can be used to monitor a patient's blood pressure and/or heart rate (30). If a reduced level of blood pressure and/or a reduced heart rate is detected (34), then a stimulatory signal can be delivered to one or more renal nerves in a manner designed to increase the patient's blood pressure (36). FIG. 4 provides an additional method that can be used to treat hypotension and syncope.

In some cases, the methods and materials provided herein can be used to treat critically ill cardiac patients with persistent postoperative vasoplegia, orthostatic syncope due to diabetic dysautonomia, other forms of reflex syncope, postural orthostatic tachycardia syndrome (POTS), and sudden cardiac death. In some cases, the methods and materials provided herein can be used treat astronauts returning from space flights who experience orthostatic intolerance and syncope.

The invention will be further described in the following examples, which do not limit the scope of the invention described in the claims.

### EXAMPLES

### Example 1

### Renal Nerve Stimulation for Treatment of Syncope

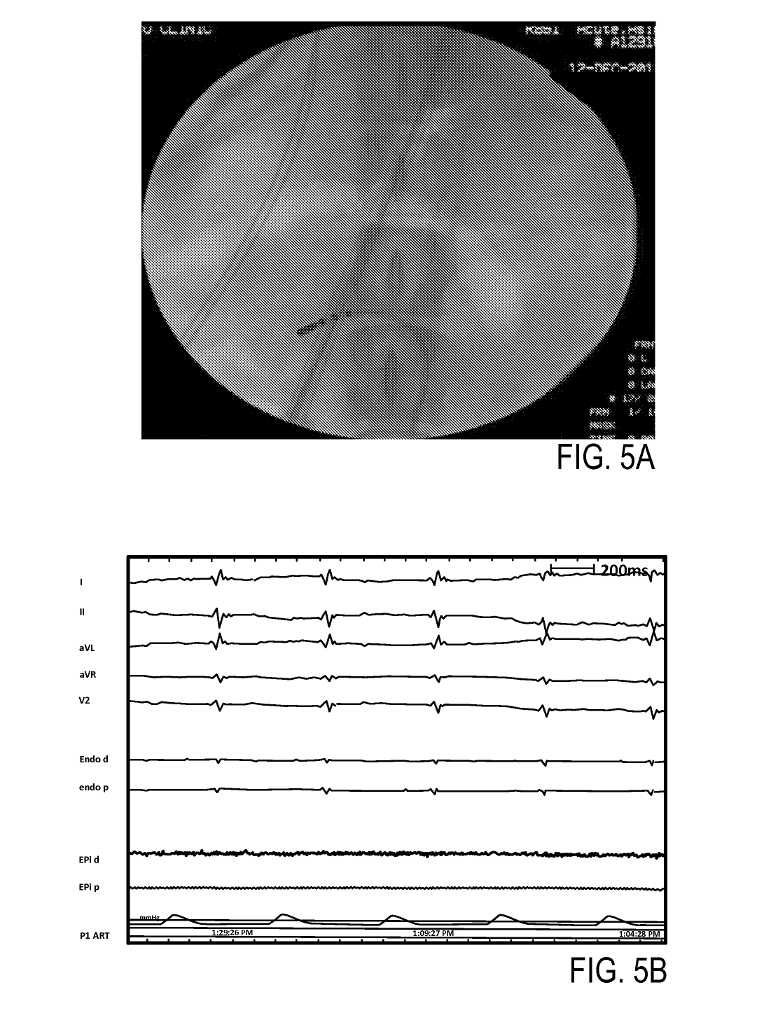

Renal nerve stimulation was performed under Isoflurane anesthesia in one dog and one baboon using a 4 mm quadripolar catheter in unilateral renal artery (A) or vein (V) using a Grass stimulator (square wave, 120V, 900 pps, 30 s, Grass Technologies) on nine occasions (FIG. 5A). A consistent increase in arterial systolic blood pressure (BP) (median (range) pre- vs. post-stimulation 99 (84-139) vs. 105 (90-165) mmHg) and diastolic BP (72 (51-89) vs. 76 (54-106) mmHg) was detected (p=0.006). The mean increase in systolic and diastolic BP was 11(±3) and 9(±3) mmHg, respectively. The BP returned to baseline within one minute of cessation of stimulation. DC ablation in canine right renal A (5000 mA, flow 60 mL/hour, 120 s, 2 applications) abolished the BP response to high rate stimulation. Thus, renal nerve stimulation through the renal vessels increased BP through sympathetic activation, and this response was abolished by ablation.

FIG. 5B is a graph plotting a recording of arterial tracing during stimulation. The blood pressure increased from a baseline of 139/80 to 163/104 following 30 seconds of stimulation.

These results demonstrate that renal nerve stimulation can be used to treat the syncope. For example, these results demonstrate that electrodes placed in the vicinity of a renal vein can result in perivenous autonomic stimulation and acute blood pressure increase. These results also demonstrate that a transvenous device can be configured with leads in the right ventricle and renal vein, and can be used to perform simultaneous cardiac pacing and renal nerve stimulation in response to a sudden drop in heart rate or BP at the beginning of a neurocardiogenic spell, thus preventing syncope.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal undergoing syncope, wherein said method comprises stimulating nerves of said mammal under conditions wherein the blood pressure of said mammal increases.

2. The method of claim 1, wherein said mammal is a human.

3. The method of claim 1, wherein said nerves are autonomic nerves in or around a renal vessel.

4. The method of claim 1, wherein said nerves are spinal nerves.

5. The method of claim 1, wherein said method comprises stimulating autonomic ganglia.

6. A method for treating syncope, wherein said method comprises:
(a) detecting a decrease in blood pressure within a mammal suffering from syncope, and
(b) stimulating nerves of said mammal under conditions wherein the blood pressure of said mammal increases.

7. The method of claim 6, wherein said mammal is a human.

8. The method of claim 6, wherein said nerves are autonomic nerves in or around a renal vessel.

9. The method of claim 6, wherein said nerves are spinal nerves.

10. The method of claim 6, wherein said method comprises stimulating autonomic ganglia.

11. An implantable device for treating syncope of a mammal, wherein said device comprises a control unit, a sensing lead for detecting the blood pressure or heart rate of said mammal, and a stimulation lead for stimulating nerves of said mammal, wherein said sensing lead and said stimulation lead are connected to said control unit, and wherein said control unit is configured to activate said stimulation lead to stimulate said nerves when said sensing lead detects a decrease in the blood pressure or heart rate of said mammal

12. The device of claim 11, wherein said mammal is a human.

13. The device of claim 11, wherein said nerves are autonomic nerves in or around a renal vessel.

14. The device of claim 11, wherein said nerves are spinal nerves.
